(19) United States
(12) Patent Application Publication
(10) Pub. No.: US 2023/0346271 A1
(43) Pub. Date: Nov. 2, 2023

(54) REMOVABLE DETECTION CARD FOR IN-VITRO MEDICAL DIAGNOSIS DEVICE AND CONTROL METHOD THEREOF

(71) Applicant: EDAN INSTRUMENTS, INC., SHENZHEN (CN)

(72) Inventors: GAOXIANG HUANG, SHENZHEN (CN); ZHIXIANG ZHAO, SHENZHEN (CN)

(21) Appl. No.: 18/343,714

(22) Filed: Jun. 28, 2023

Related U.S. Application Data
(63) Continuation of application No. PCT/CN2021/143210, filed on Dec. 30, 2021.

(30) Foreign Application Priority Data
Dec. 31, 2020 (CN) 202011629595.9

(51) Int. Cl. A61B 5/1455; A61B 5/15; A61B 5/157

(52) U.S. Cl. CPC A61B 5/14552; A61B 5/150343; A61B 5/157

(57) ABSTRACT

The present application provides a removable detection card for an in-vitro medical diagnosis device. The removable detection card at least includes internal liquid paths, a blood gas test area, a hemoglobin and derivative thereof test area, a liquid path control area and a waste liquid area. The blood gas test area and the hemoglobin and derivative thereof test area are distributed on different liquid paths, so that the same blood sample or different blood samples can enter the blood gas test area or the hemoglobin and derivative thereof test area by controlling, by the liquid path control area, switching between the internal liquid paths of the removable detection card, the use cost of the detection card is reduced, and moreover, the calibration precision of the detection card can be ensured.

### CROSS-REFERENCE TO RELATED APPLICATIONS

The present application is a continuation of International (PCT) Patent Application No. PCT/CN2021/143210 filed on Dec. 30, 2021, which claims priority to Chinese patent application No. 202011629595.9, filed on Dec. 31, 2020, the contents of all of which are hereby incorporated by reference.

### TECHNICAL FIELD

The present disclosure relates to the field of in-vitro technologies, and more particularly, to a detection card for use in-vitro medical diagnosis device and a method for controlling the detection card.

### BACKGROUND

The determination of gas component in blood is very important in various scientific researches and practical applications. In the rescue of critical clinical medicine patients, rapid and continuous determination of the partial pressure of blood carbon dioxide is critical. Especially for mechanically ventilated patients, blood carbon dioxide partial pressure is a key index for judging respiratory state of patient, and various parameters of the breathing machine are mainly set according to the partial pressure of carbon dioxide in the blood of the patient. At present, the most widely used blood gas component detector in medicine is a blood gas analyzer, but the conventional blood gas analyzer has the defects that a large number of blood samples need to be collected, the detection is discontinuous, the detection result is lagged, and the like.

The traditional in-vitro blood gas detection is performed in a large test center with good equipment. Although these conventional test centers can provide efficient and accurate testing of large volume fluid samples, it is not possible to provide a direct result. The practitioner must collect the fluid sample, send it to the laboratory, then the fluid sample is processed by the laboratory, and finally, the result is conveyed the patient. This traditional detection method causes a long and complex cycle of blood gas testing, it is difficult for patients to obtain timely diagnostic results, which is not conducive to timely diagnosis by medical staff and cannot provide patients with a good medical experience.

In addition, conventional in-vitro diagnostic testing requires training laboratory technicians to perform the test, thereby ensuring the accuracy and reliability of the test. However, usage errors caused by personnel handling samples may lead to surface contamination, sample spillage, or damage to diagnostic devices, resulting in increased repair and maintenance costs.

At the same time, in the in-vitro diagnostic apparatus of the related art, if only one detection card is used to detect the blood of the patient, only blood gas detection is completed, or only, hemoglobin detection is completed. If the blood of the patient needs to detect the indexes of blood gas and hemoglobin at the same time, it is often necessary to take two copies of the patient's blood. This is not a common problem for healthy adults, however; however, for newborns or patients with less blood volume (e.g., anemia), various inconveniences may be brought to them. At the same time, even for healthy adults, if the indexes of blood gas and hemoglobin need to be detected simultaneously, two detection cards are needed, and the diagnosis cost of the patient is greatly increased. It is also possible to cause waste of resources.

At the same time, in the related art, it is often necessary to calibrate the sensors in the detection card before using the detection card. Therefore, in order to save cost, the calibration liquid needed by calibration is usually stored in the detection card. However, the consequent problem is that if the calibration liquid is stored in the detection card, in order to ensure that the calibration liquid is not affected by adverse environmental factors such as temperature and humidity, the detection card requires high storage conditions and environmental requirements during transportation and storage, to prevent the calibration liquid inside of the detection card from being affected, which in turn affects the calibration of the detection card sensor.

### SUMMARY OF THE DISCLOSURE

A removable detection card for an in-vitro medical diagnosis device, includes a blood gas detection area, a hemoglobin and its derivatives detection area, an internal pipeline, and at least two external interfaces including a calibration liquid inlet and a sample to he detected inlet. The detection card is configured to allow a calibrating liquid required by the detection card to enter the detection card through the calibration liquid inlet after being mounted in a detection area of an in-vitro medical diagnosis device,

in some embodiments, one or more first cavities for blood gas detection is located in the blood gas detection area, and one or more second cavities for detecting a hemoglobin and its derivatives is located in the hemoglobin detection area.

In some embodiments, the removable detection card further includes a photochemical sensor or an electrochemical sensor located in the one or more first cavities.

In some embodiments, the one or more second cavities are adapted for detecting the hemoglobin and its derivatives by colorimetric or electrochemical method.

In some embodiments, the second cavity is thinner than the first cavity.

in some embodiments, a diameter of the second cavity is greater than a diameter of the first cavity.

In some embodiments, the first cavity has a thickness of 0.6 mm to 0.9 mm, and/or the second cavity has a thickness of 0.1 mm to 0.5 mm.

In some embodiments, a distance between the one or more first cavities is at least greater than 7 mm.

In some embodiments, the one or more first cavities have an interior width of 2.5 mm, 3.5 mm, or 4.5 mm.

In some embodiments, a liquid path width between the one or more first cavities is 1 mm.

In some embodiments, the removable detection card further includes a waste liquid area, wherein the internal pipeline is configured to provide a flow path between the blood gas detection area, the hemoglobin and its derivatives detection area, and the waste liquid area.

In some embodiments, the removable detection card further includes a first control part configured to control on-off between the waste liquid area and the internal pipeline.

In some embodiments, the removable detection card further includes a second control part configured to control the on-off between the sample to be detected inlet and the internal pipeline.

In some embodiments, the removable detection card further includes a third control part configured to control the on-off between the internal pipeline and the hemoglobin and its derivatives detection area.

In some embodiments, the removable detection card further includes an elastomeric material and/or valve inside or on a surface of the detection card the elastomeric material and/or valve is configured to implement on-off.

In some embodiments, the waste liquid zone has an external interface for communicating with ambient air pressure.

In some embodiments, the external interfaces are configured to be distributed on the same or different sides of the detection card.

In some embodiments, the detection card includes at least two control parts; in an initial state, the two control parts are configured to enable the pipelines corresponding to the two control parts to be in a cut-off state; and after the detection card enters an operation state, the two control parts are configured to enable the pipelines corresponding to only one control part to be in a conduction state at the same time.

In some embodiments, a first control part controls on-off between the waste liquid area and the internal pipeline in response to calibrating the sensor in the detection card or requiring discharge of the calibration liquid to the waste liquid area.

In some embodiments, a second control part controls on-off between the sample to be detected inlet and the internal pipeline in response to injecting a sample to be detected into the detection card.

In some embodiments, a third control part controls on-off between the internal pipeline and the hemoglobin and its derivatives detection area, in response to transferring the sample to be detected in the blood gas detection area to the hemoglobin and its derivatives detection area.

In some embodiments, each control part of the detection card controls on-off of a corresponding pipeline in response to a power drive in a host of the in-vitro medical diagnosis device.

A detection method for a removable detection card, includes: a detection card including a control unit, wherein an internal pipeline is configured to provide a flow path between a blood gas detection area and a hemoglobin and its derivatives detection areas; in response to a first state of the control unit, a first sample to be detected enters the blood gas detection area; and in response to a second state of the control unit, a second sample to be detected enters the hemoglobin and derivative detection area.

In some embodiments, the first sample to be detected and the second sample to be detected are the same sample to be detected.

In some embodiments, the detection card further includes a waste liquor area, the internal pipeline is configured to provide the flow path between the blood gas detection area, the waste liquor zone, and the hemoglobin and its derivatives detection area.

In some embodiments, removable detection card further includes: in response to a third state of the control unit, a calibration liquid enters the blood gas detection area for sensor calibration, and the calibration liquid is transferred to the waste liquid area after completing calibration.

In some embodiments, the second sample to be detected enters the hemoglobin and its derivatives detection area, and the second sample to be detected is transferred to the waste liquid area after the hemoglobin and its derivatives detection is completed.

In some embodiments, the control unit includes at least three control parts configured to be driven by a host of an in-vitro medical diagnosis device.

In some embodiments, a first control part of the at least three control parts controls on-off between the waste liquid area and the internal pipeline of the detection card, a second control part controls on-off between a sample to be detected inlet and the internal pipeline, and a third control part controls the on-off between the internal pipeline and the hemoglobin and its derivatives detection area.

In some embodiments, the first state is that the second control part is in conduction, and the first control part and the third control part are both in turn-off state, to make the sample to be detected inlet and the internal pipeline be conducted; and the detection card is configured to be provided with a negative pressure by a reagent pack that is connected to the detection card through a calibration liquid inlet, to suck the sample to be detected into the blood gas detection area.

In some embodiments, the second state is that the third control part is in conduction, and the first control part and the second control part are in turn-off state, so that the internal pipeline is communicated with the hemoglobin and its derivatives detection area; and the detection card is configured to be provided with a positive pressure by a reagent pack that is connected to the detection card through a calibration liquid inlet, to transfer the sample to be detected in the blood gas detection area to the hemoglobin and its derivatives detection area.

In some embodiments, the control unit has a third state, the third state is that the first control part is in conduction and the first control part and the second control part are both in the off state, so that the waste liquid area is communicated with the internal pipeline; and the detection card is configured to be provided with a positive pressure by a reagent pack that is connected to the detection card through a calibration liquid inlet, the calibrating, liquid in the reagent pack is transferred to the blood gas detection area through the calibration liquid inlet, and after calibration, the reagent pack provides the positive pressure again to transfer the calibrating liquid in the blood gas detection area to the waste liquid area.

In some embodiments, the blood gas detection area is provided with one or more photochemical or electrochemical sensors adapted for blood gas detection.

In some embodiments, the hemoglobin and its derivatives detection area is provided with a cavity adapted to detect hemoglobin and its derivatives by colorimetric or electrochemical method.

### BRIEF DESCRIPTION OF THE DRAWINGS

FIG. 1 is a schematic view of an external structure of an in-vitro medical diagnostic system.

FIG. 2 is a schematic side view of the in-vitro medical diagnostic system.

FIG. 3 is a schematic view of a combination of components of the in-vitro medical diagnostic system.

FIG. 4 is a schematic semi-sectional view of a removable detection card.

FIG. 5 is another schematic semi-sectional view of the removable detection card.

FIG. 6 is yet another schematic semi-sectional view of the removable detection card.

FIG. 7 is a schematic view of the connection of the removable detection card and a valve control device.

FIG. 8A is a schematic control view of the valve control device.

FIG. 8B is another schematic control view of the valve control device.

FIG. 8C is yet another schematic control view of the valve control device.

FIG. 8D is yet another schematic control view of the valve control device.

FIG. 9 is a schematic cross-sectional view of a removable reagent pack.

FIG. 10 is a schematic view of an external structure of the removable reagent pack.

FIG. 11 is a schematic view of a body and a decoration cover of the removable reagent pack.

FIG. 12A is a schematic view of an excitation light source of the in-vitro medical diagnostic system.

FIG. 12B is another schematic view of an excitation light source of the in-vitro medical diagnostic system.

### DETAILED DESCRIPTION

The present disclosure will now be further described in conjunction with the accompanying drawings and specific embodiments.

Before turning to the drawings illustrating exemplary embodiments in detail, it is to be understood that the present disclosure is not limited to the details or methods shown in the specification or illustrated in the drawings. The purpose of professional terminology is only for illustration and should not limit the understanding of present product and corresponding methods.

In view of the foregoing, a purpose of the present disclosure is providing a removable detection card for use in an in-vitro diagnostic device, and the removable detection card is capable of at least partially mitigating or eliminating at least one defect in the related art.

It is necessary to design a removable detection card for an extracorporeal medical diagnosis device, capable of completing detection of blood gas and hemoglobin indexes in one detection. card, and more ideally, completing detection of blood gas and hemoglobin indexes in one detection card by using the same blood sample.

It is necessary to design a detection card that is less affected by environmental factors during transportation and storage, to reduce the cost of using the detection card and ensure the accuracy of the detection results.

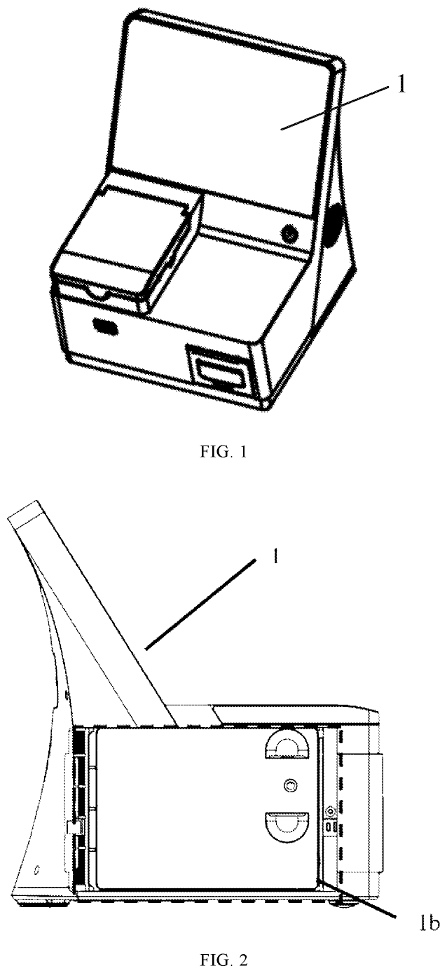

Exemplary embodiments of the present disclosure provide an in-vitro medical diagnostic system. As shown in FIGS. 1-3, the in-vitro medical diagnostic system includes a host 1. removable detection card 2, and a removable reagent pack 3. The host 1 includes a housing, and processing circuitry, a power supply circuitry and optical elements located in the housing. The housing further includes a first area 1a configured to at least partially accommodate the removable detection card 2 and a second area 1b configured to at least partially accommodate the removable reagent pack 3. The removable detection card 2 is jointed with the host 1 and the removable reagent pack 3 through the first area 1 a and the second area 1b, respectively, such that there is no fluid communication between the removable detection card 2 and the host 1, and there is no fluid communication between the removable reagent pack 3 and the host 1.

### Removable Detection Card

The removable detection card 2 includes an external interface, a detection area, a waste liquid area, a liquid path control area and an internal liquid path.

The external interface refers to a first interface for receiving fluid samples, a second interface for air pump inlet/outlet, and a third interface for injecting calibration liquid located on the sides, top, or bottom of the detection card 2. Specifically, a fluid sample inlet is on the first side of the detection card 2. The fluid sample inlet is configured to receive fluid samples, such as whole blood samples that do not require hemolysis. A calibration liquid inlet and an exhaust port are on the second side of the detection card 2. The calibration liquid inlet is configured to receive calibration liquid from the outside (such as the removable reagent pack 3) for calibration of various photochemical sensors inside the detection card 2. The exhaust port is configured to be connected to the external atmosphere to maintain pressure balance in the detection card 2, in order to ensure that the calibration liquid or fluid sample flows into the detection card 2.

The detection area refers to the area where electrical, optical, chemical sensors and/or cavities without sensors are placed for detecting various blood gas parameters. The detection of blood gas, hemoglobin, electrolytes, and other biochemical parameters is completed in this area.

The waste liquid area refers to the area configured to store the detected fluid samples.

The internal liquid path refers to the pipeline path configured to allow the fluid sample or calibration liquid to flow inside of the detection card 2. The pipeline path has multiple interconnected liquid paths, and the detection area, the waste liquid area, and the liquid path control area are located on different liquid paths.

The liquid path control area refers to the area where several on-off control devices are located. The on-off control devices are configured to control the on-off of the internal liquid path, thereby achieving liquid path switching, allowing the calibration liquid and fluid samples at different testing stages to flow into the detection area and waste liquid area through different paths, The detailed switching operation will be described in detail later.

The detection area, the waste liquid area and the liquid path control area are generally located between the first side and the second side of the detection card 2.

Each functional area and the internal liquid path of the detection card 2 have a plurality of implementation modes, and one of the preferred implementation modes is provided as follows.

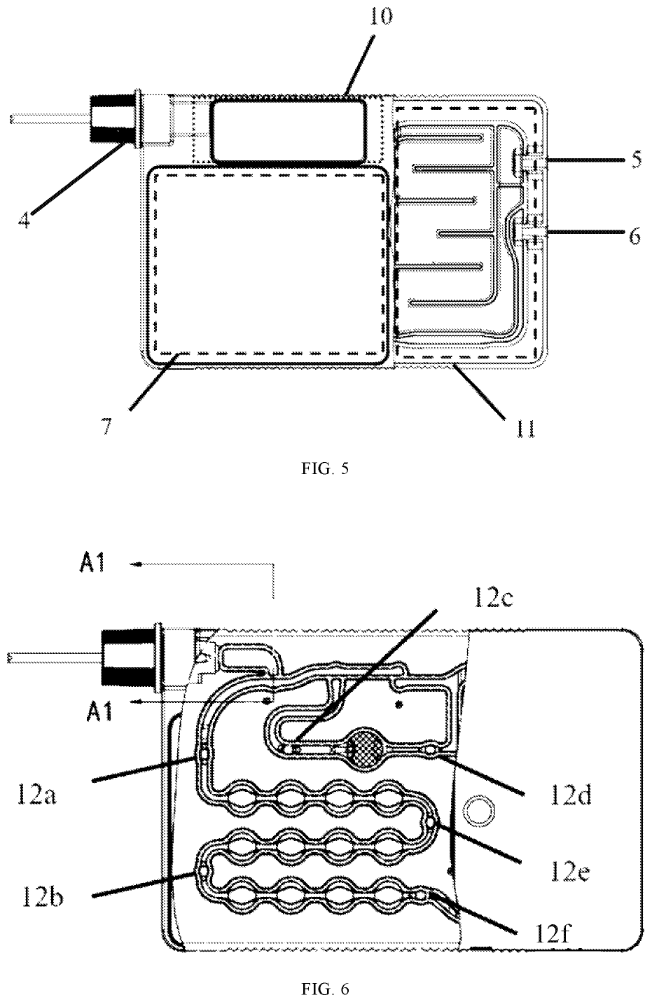

As shown in FIGS. 4-6, the removable detection card 2 includes a card body, at least one portion of the card body is transparent. The card body may be made of molded plastic, additional material, or sets of materials. The parameters such as transmittance and refractive index of the card material for the corresponding wavelength of light need to meet the optical detection. requirements of in vitro medical diagnostic systems. In order to better reflect the division of the transparent housing and internal functional areas of the card body, the semi-sectional views in FIGS. 4-6 are used to show the housing and internal functional areas of the detection card 2.

The fluid sample to be detected is a blood sample. In an embodiment, the fluid sample is a whole blood sample without hemolysis. The detection area includes a blood gas detection area 7 and a hemoglobin and its derivatives detection area 8. The waste liquid area 11 is configured to store the detected blood sample. The internal liquid path 9 is divided into a main liquid path and three controllable liquid paths. The main liquid path is connected to the calibration liquid inlet 6, the blood gas detection area 7 and the liquid path control area 10. In an embodiment, the blood gas detection area 7 is between the calibration liquid inlet 6 and the liquid path control area 10. The first controllable liquid path is configured to control the on-off of the liquid path between the sample inlet 4 and the main liquid path. The second controllable liquid path is configured to control the on-off of the liquid path between the main liquid path and the inlet of the hemoglobin. and its derivatives detection area 8, and the outlet of the hemoglobin and its derivatives detection area 8 is communicated to the waste liquid area 11. The third controllable liquid path is configured to on-off the waste liquid area 11 and the main liquid path. The waste liquid area 11 is connected. to the exhaust port 5. The blood gas control area and the second controllable liquid path are arranged with several position detection points. The liquid path control area 10 is provided with three valves 10a to 10c for controlling the on-off of the internal liquid path 9, respectively configured to control the first, second and third controllable liquid paths. The control mode will be described in detail in the following sections.

The blood gas detection area 7 has twelve sensor cavities defined as 7A to 7L, the cavities are sequentially arranged on the main liquid path. The cavity may be of various shapes, and the shapes of the cavities can be the same or different. However, in the flow direction of the liquid path, the width of the sensor cavity is wider than the width of the liquid path. Various types of sensors can be placed in the cavity. The twelve sensor cavities are arranged from far to near according to the distance from the calibration liquid inlet in the direction of the flow path, sequentially numbered 7A-7L. The first eleven sensor cavities 7A-7K are sequentially provided with different photochemical sensors, the twelfth sensor cavity 7L is used as a standby for future detection parameter expansion, and the hemoglobin and its derivatives detection area 8 is only a cavity and is not provided with a sensor.

In an embodiment, the distance between adjacent cavities 7A to 7L in the blood gas detection area 7 is at least greater than 7 mm. Each of the cavities 7A to 7L has an internal width of 2.5 mm, 3.5 mm or 4.5 mm. The numerical setting aims at effectively preventing the incident and reflected light of the adjacent cavities from interfering with each other during optical detection, so that the distance and width of the adjacent cavities need to be matched to ensure that the optical paths of the adjacent cavities do not interfere with each other. The numerical values can be correspondingly adjusted according to different sizes of the optical fibers.

in an embodiment, the width of the liquid path between the cavities 7A to 7L, in the blood gas detection area 7 is 1 mm, which can be adjusted according to the viscosity of the sample blood,

In an embodiment, the photochemical sensors in each of the first ten sensor cavities 7A-7J are configured to detect the blood gas parameters in the blood fluid sample, such as CO₂, O₂, pH, Na⁺, M⁺⁺, or the like.

In an embodiment, the fluid sample can be a whole blood sample, a urine sample and other types of human body fluid samples, and in this case, the sensor in the detection card 2 detects corresponding biochemical parameter indexes.

The control mode of the detection card 2 is described in detail below.

Before detecting the blood samples, it is necessary to calibrate all sensors in detection card 2, that is, inject calibration liquid into the sensor, empty all calibration liquid in detection card 2 after calibration, and then inject fluid samples to be detected. In an embodiment, the fluid samples are blood samples that do not require hemolysis, The testing is completed in the detection area, and the corresponding parameters are read through the host of the in-vitro medical diagnosis system. After completing the diagnostic analysis, the corresponding parameters and diagnostic results are displayed on the host screen, so that medical personnel can timely learn about the corresponding situation.

The specific operation steps are as follows:
operation S1: placing the detection card 2 into the first area 1a;
operation S2: pumping the calibration liquid into the detection area in the detection card 2;
operation S3: after finishing calibration, transferring the calibration liquid in the detection card 2 to the waste liquid area 11 in the detection card 2;
operation S4: injecting blood sample into the detection area to complete the detection of at least blood gas and hemoglobin and its derivatives in the detection area; and
operation S5: transferring the blood sample in the detection area to the waste liquid area 11 in the detection card 2 to complete the detection.

The operation S1 specifically includes as follows. Before the detection card 2 is jointed with the reagent pack 3 and the host 1, the detection card 2 is internally dry and has no calibration liquid. The calibration liquid is stored in the reagent pack 3, separated from the detection card 2. The detection position is the first area 1a in the host 1. When the detection card 2 is placed in the first area 1a, the fluid sample in the detection card 2 is detected. As shown in FIG. 6, at least six position detection points 12a to 12f are arranged in the detection card 2 and can receive light intensity emitted by a detection light source and transmitted through the detection card 2, so that whether liquid exists at the position detection points 12a to 12f or not can be detected and judged.

The operation S2 specifically includes as follows. The third controllable liquid path is communicated, the first and second controllable liquid paths are controllably closed. The host 1 controls the peristaltic pump 16 and the three-way valve 17 in the reagent pack 3, switching the three-way valve 17 to the calibration liquid pipeline 19, pumping the calibrating, liquid (i.e., standard sample liquid with known composition and concentration) in the calibration liquid bag 15 into the detection card 2 through a connecting piece 20 inserted into the calibration liquid inlet of the detection card 2. When the position detection point 12a detects that liquid exists, the host 1 controls the peristaltic pump 16 in the reagent pack 3 to stop moving, at the moment, the sensor cavities 7A to 7K are filled with the calibration liquid. The host 1 starts calibrating the sensors (i.e., reading the readings of the liquid sensors of the standard sample measured by the sensors), and the host 1 finishes calibrating after reading the detection values of the sensors.

The operation S3 specifically includes as follows. The third controllable liquid path is kept communicated, and the first and second controllable liquid paths are kept closed. The host 1 controls the three-way valve 17 in the reagent pack 3 to be switched to the air channel. The peristaltic pump 16 is controlled to pump the air into the detection card 2 through the connecting piece 20 inserted into the calibration liquid inlet of the detection card 2. Because the waste liquid area 11 of the detection card 2 is connected to the outside air through the exhaust port 5, with the pumping of the air, the calibration liquid continuously moves to the waste liquid area 11 in the first and third liquid paths until all the calibration liquid enters the waste liquid area 11.

The operation S4 includes two stages, i.e., a first stage and a second stage as follows.

The blood gas detection is performed in the first stage. Specifically, the first controllable liquid path is communicated, the second and third controllable liquid paths are closed, and the blood gas detection is performed in this case. The blood sample is injected from the sample inlet 4 of the detection card 2, without hemolysis, or the host 1 controls the peristaltic pump 16 to rotate reversely, extracting the air in the detection card 2, negative pressure is generated in the detection card 2 to suck blood at the sample inlet 4 into the detection card 2. Thus, after it is determined by the position detection points 12a to 12f that the blood sample to be detected completely enters the sensor cavities 7A to 7L, stopping injecting the blood sample to be detected into the detection card 2, alternatively, the host 1 controls the peristaltic pump 16 to stop pumping air, and the blood sample is detected by using the sensor cavities 7A to 7L. In an embodiment, the photochemical sensors are arranged in the sensor cavities 7A to 7K, the sensor cavity 7L is used as standby sensors or other types of sensors are placed in the sensor cavity 7L.

The basic detection principle of the photochemical sensor is that the photochemical method uses organic dyes to emit fluorescence with different wavelengths from the irradiation light under specific wavelengths of light, influenced by substances such as O₂ and CO₂ concentration and pH. The fluorescence is transmitted to the detector to detect its fluorescence signal and perform. quantitative analysis, thereby being able to detect O₂, CO₂, and pH values.

Entering the second stage after completing blood gas detection, that is, hemoglobin and its derivatives are detected. The first controllable liquid path is kept communicated and the third controllable liquid path is kept closed, the second controllable liquid path is opened. The host controls the peristaltic pump 16 to rotate forward, pumping air into the detection card 2, so that the blood in the blood gas detection area is sent into the hemoglobin and its derivatives detection area 8 through the second controllable liquid path. Then the host 1 controls the peristaltic pump 16 to stop pumping air into the detection card 2, using the light emitted from the first light source to irradiate the hemoglobin and its derivatives detection area 8 from one side, and receiving the transmitted light from the other side of the hemoglobin and its derivatives detection area 8 to detect whether hemoglobin and its derivative are present in the blood sample or not according to the detection principle of colorimetric method.

In some embodiments, it can be considered that, before entering the first stage, the detection card 2 is in an initial state. After entering the first stage, the detection card 2 is in an operating state.

The operation S5 specifically includes as follows. After completing the detection of hemoglobin and its derivative, the host 1 controls the peristaltic pump 16 to rotate forward and pumps air into the detection card 2 to push the blood sample in the hemoglobin and its derivatives detection area 8 into the waste liquid area 11. After the blood sample enters the waste liquid area. 11, the host 1 controls the peristaltic pump 16 to stop rotating and the test is finished.

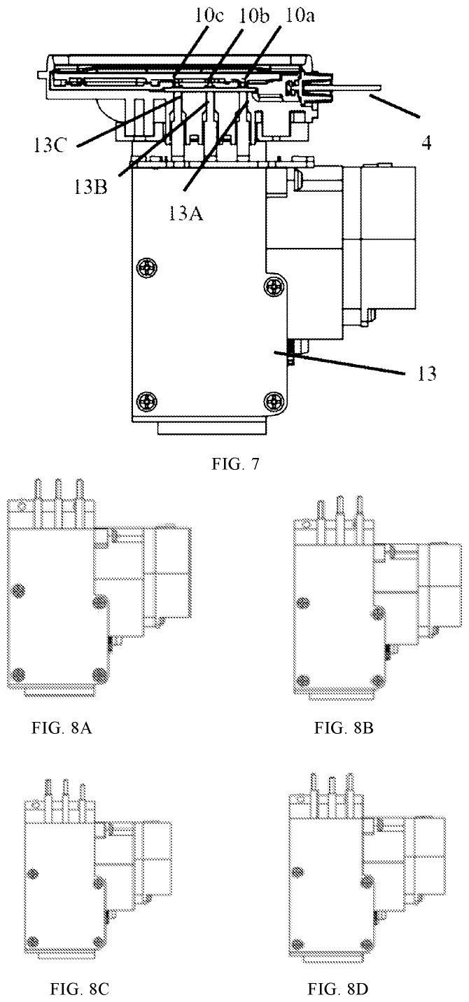

In the above operation steps, the control of the first, second and third controllable liquid paths is realized as shown in FIG. 7 and FIGS. 8A-8D.

Three valves 10a to 10c are inside of the liquid path control area 10. The valves 10a to 10c are respectively configured to control the on-off of the first, second and third controllable liquid paths, and the on-off of the valves 10a to 10c, are respectively driven by the control mechanisms 13A to 13C in the valve control device 13. When one of the control mechanisms 13A to 13C moves downwards, one corresponding valve in the valves 10a to 10c is opened, and the corresponding controllable liquid path is conductive. Otherwise, when one of the control mechanisms 13A to 13C moves upwards to the end, one corresponding valve in the valves 10a to 10c is closed, and the corresponding controllable liquid path is not conductive.

Specifically, in the operation S1, the valves 10a to 10c are all in an off state, and the positions of the control mechanisms are shown in FIG. 8A, that is, the first, second and third controllable liquid paths are all disconnected.

In the steps S2 and S3, the valves 10a to 10b are in a closed state, the valve 10c is in a conductive state, the positions of the control mechanisms are shown in FIG. 8B, and the main liquid path is connected to the waste liquid area 11, so that the calibration liquid can he pumped into the detection card 2. After completing calibration, air is pumped into the detection card 2, so that the calibration liquid enters the waste liquid area 11.

In the first stage of the operation S4, the valve 10a is controlled to be conductive, the valves 10b to 10c are in the closed state, the positions of all the control mechanisms are shown in FIG. 8C, and the main liquid path is connected to the sample inlet 4, so that the blood sample can be injected into the detection card 2, and the sample blood reaches the blood gas detection area 7 for blood gas detection.

In the second stage of operation 84, that is, after the blood gas detection is completed, the valve 10b is controlled to be conductive, and the valves 10a and 10c are in the off state. The positions of the control mechanisms are shown in FIG. 8D, the main liquid path is connected to the hemoglobin and its derivatives detection area 8, so that the blood sample can enter the hemoglobin and its derivatives detection area 8, and the detection of the hemoglobin and its derivatives is performed.

After the detection is completed, the blood sample is sent into waste liquid area 11..

The turn-off sequence of the valves 10a to 10c may he set to a fixed timing sequence or customized and logically programmed according to the needs of the operator.

In an exemplary embodiment, the widths of the first, second and third liquid paths may be set to he different. In an embodiment, the width of the second liquid path is greater than the width of the first liquid path. The thickness of the sensor cavity of the detection card 2 in the blood gas detection area 7 is different from the thickness of the cavity in the hemoglobin and its derivatives detection area 8, so as to meet the requirements of blood gas photochemical detection of whole blood sample and different detection methods of hemoglobin and its derivatives,

In an exemplary embodiment, the detection card 2 is configured to be discarded after using. Alternatively, the detection card 2 can be configured to be recycled to test more than one fluid sample.

Furthermore, an electrochemical sensor can be used in the detection card 2 to detect blood gas, hemoglobin and its derivatives. The electrochemical detection technology is mature, which is not required to be described in detail.

### Reagent Pack

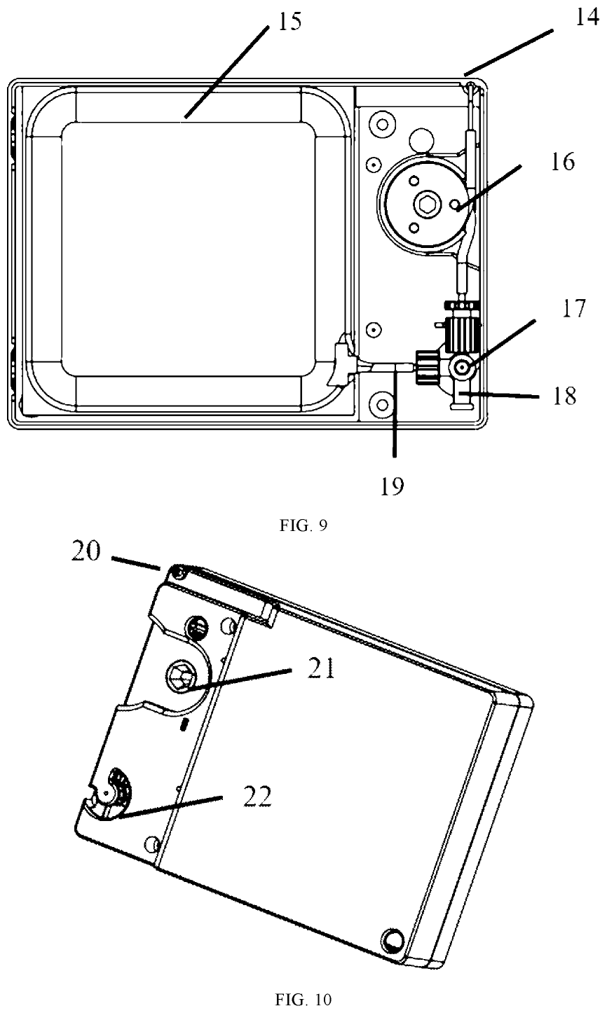

As shown in FIG. 9, FIG. 9 is a schematic cross-sectional view of the reagent pack 3. The reagent pack 3 may he disposable and removable. The reagent pack 3 includes a housing 14, a calibration liquid bag 15, a peristaltic pump 16, a three-way valve 17, an air pipeline 18, and a calibration liquid pipeline 19, a connecting piece 20, a pump interface 21, and a valve connecting piece 22. The calibration liquid bag 15, the peristaltic pump 16, the three-way valve 17, the air pipeline 18, and the calibration liquid pipeline 19 are located in the housing 14. The connecting piece 20 is inserted into the calibration liquid inlet 6 of the detection card 2. The pump interface 21 is connected to the shaft of the stepper motor of the host 1. The stepper motor drives the peristaltic pump 16 of the reagent pack 3 to forward rotate or reversely rotate through the pump interface 21. The host 1 controls the three-way valve 17 of the reagent pack 3 to switch through the valve connecting piece 22, thereby connecting the pump interface 21 to the air pipeline 18 or the calibration liquid pipeline 19. The calibration liquid bag 15 is configured to store the calibration liquid and is configured to be connected to the three-way valve 17 through the calibration liquid pipeline 19.

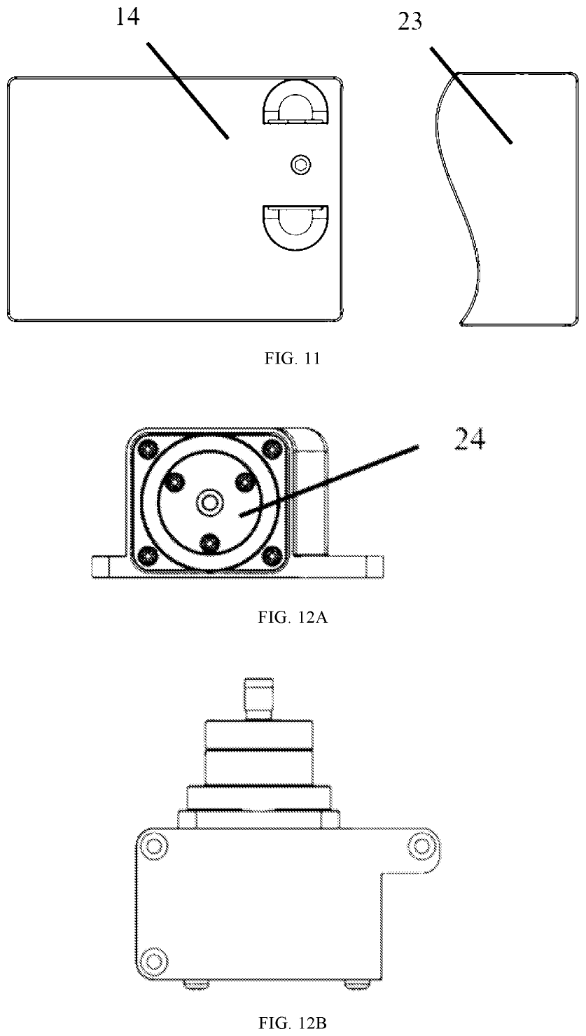

In an exemplary embodiment, the housing 14 of reagent pack 3 is made of plastic, and can also be made of other materials or sets of materials. The reagent pack 3 can also include a decoration cover. As shown in FIG. 11, the decoration cover 23 of the reagent pack 3 is connected to the front part of the housing 14 of the reagent pack 3. When reagent pack 3 is not in use (i.e., not connected to the host 1), the decoration cover 23 of the reagent pack 3 can protect the housing 14 of reagent pack 3. The calibration liquid bag 15 can be an unused soft elastic fluid bag filled with the calibration liquid.

Before the detection card 2 and the reagent pack 3 are respectively, jointed with the host 1, the interior of the detection card 2 is dry and has no calibration liquid, and the calibration liquid is completely stored in the reagent pack 3 and is separated from the detection card 2.

When the detection card 2 is placed in the first area 1a, the connecting piece 20 on the reagent pack 3 is inserted into the calibration liquid inlet 6 of the detection card 2. In an embodiment, the connecting piece 20 is a tubular steel needle and the circumference of the tubular steel needle is provided with a sealing ring, such as a rubber sealing ring, in order to ensure the sealing performance of the inserting position of the tubular steel needle and the calibration liquid inlet 6 of the detection card 2. After the calibration liquid is pumped into the detection card 2, the calibration liquid cannot leak from the calibration liquid inlet 6. The controller in the host 1 controls the rotating shaft of the stepper motor to output power, and the peristaltic pump 16 in the reagent pack 3 is controlled to forward rotate or reversely rotate through the pump interface 21, so that the detection card 2 can complete calibration and fluid sample detection, and the specific working principle is described in detail below.

When the detection card 2 and the reagent pack 3 are respectively arranged in the first area 1a and the second area 1b of the host 1, the working principle of the reagent pack 3 is as follows.

When the calibration liquid in the reagent pack 3 needs to be output, such as, in the calibration stage of the detection card 2 in the operation S2, the three-way valve 17 in the reagent pack 3 is switched to the calibration liquid pipeline 19 to he connected to the connecting piece 20. The peristaltic pump 16 is in a forward rotation state, so that the reagent pack 3 can output the calibration liquid outwards, and after the calibration liquid enters the detection card 2, the photochemical sensor in the detection card 2 is calibrated.

When air needs to he pumped to the outside. for example, when the calibration liquid and the detected blood sample are transferred to the waste liquid area 11 of the detection card 2 in the operation S3 and the operation S5, or when the blood sample is transferred to the hemoglobin and its derivatives detection area $ after the blood gas detection is completed in the second stage of operation S4, the three-way valve 17 in the reagent pack 3 is switched to the air pipeline 18 to be connected to the connecting piece 20, the peristaltic pump 16 is in the forward rotation state, so that the reagent pack 3 can pump air into the detection card 2, and the blood sample in the detection card 2 can flow in the liquid path conducted in the detection card 2 with the pumping of the air.

When air needs to be sucked into the reagent pack 3, for example, in the first stage of the operation S4, when the blood sample is sucked into the blood gas detection area 7 in the detection card 2, the three-way valve 17 in the reagent pack 3 is switched to the air pipeline 18 to be connected to the connecting piece 20. The peristaltic pump 16 is in a reverse rotation state, so that the air in the detection card 2 is sucked into the reagent pack 3, and the Hood sample enters the liquid path in the detection card 2 under the action of air pressure, thereby performing the blood gas detection.

### Host

As previously described, the host 1 includes the housing, and the processing circuitry, the power supply circuitry, and the optical clement are located in the housing. The housing may be plastic or any other material suitable for use in the present disclosure. The housing includes the first area 1a configured to at least partially accommodate the removable detection card 2, and the second area 1b configured to at least partially accommodate the removable reagent pack 3. The removable detection card 2 is jointed with the host 1 and the removable reagent pack 3 through the first area 1a and the second area 1b, respectively, thereby performing blood gas detection, hemoglobin and its derivatives detection or other biochemical parameter detection. The detection. card 2 and the host 1 only have mechanical transmission connection, no liquid path connection. Only mechanical power transmission connection exists between the reagent pack 3 and the host 1, there is no liquid path connection between the reagent pack 3 and the host 1. The detection card 2 is connected to the reagent pack 3 through the calibration liquid inlet 6, and calibration liquid flowing and air flowing are achieved. Due to above design mode, a liquid path system does not exist in the host 1, and liquid path flowing with the detection card 2 and the reagent pack 3 is not needed.

In an exemplary embodiment, the first area 1a of the housing includes a test slot for accommodating the detection card 2 containing the fluid sample, such as the blood sample. The syringe is configured to be connected to the sample inlet 4 of the detection card 2. The host 1 is configured to test the fluid sample and report the result to the user through the output unit, for example, host 1 may include a display serving as the output unit. However, in this embodiment or other embodiments, diagnostic results may also or alternatively be reported to the user by other output units, including an audio output unit, a data communication output unit, or a print output unit, and so on.

In an exemplary embodiment, once the fluid sample is tested, the detection card 2 may be removed from the host 1. The host 1 may include an ejection button, once the test is complete, the user can depress the ejection button to eject the detection card 2 from the test slot. When the test cycle is completed, the host 1 may also be configured to automatically eject the detection card 2. In an embodiment, the host 1 may be portable.

In an exemplary embodiment, the diagnostic results are displayed on the display. The processing circuitry of host 1 may cause the display to show information relating to a particular application. The display may be a unidirectional screen configured to display the output to the user, alternatively, the display may he a touch screen configured to receive and respond to the user's touch input. In an exemplary embodiment, the diagnostic device further includes a print slot configured to receive paper output by the printer housed within the host 1.

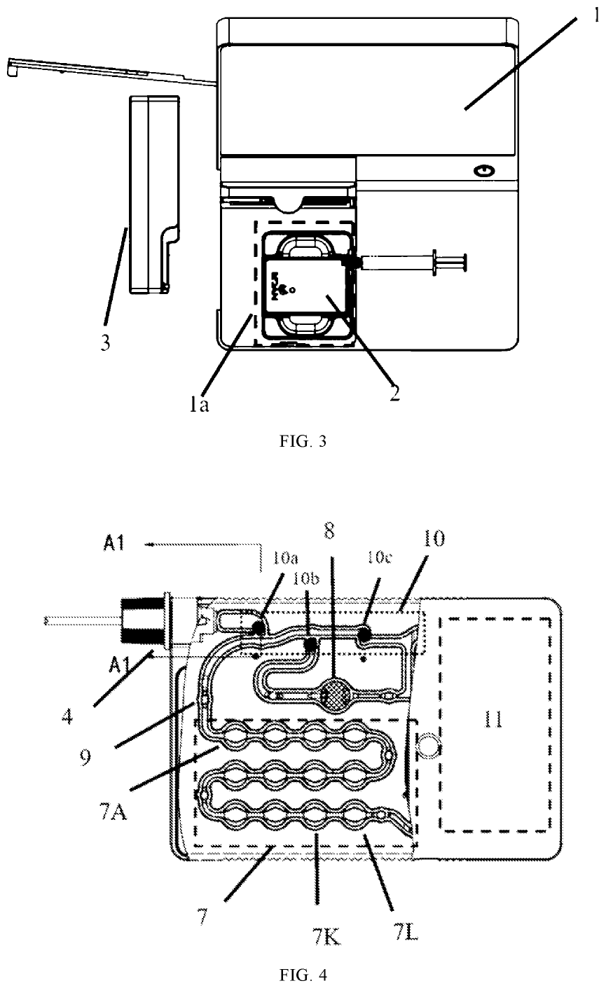

In an exemplary embodiment, as shown in FIG. 3, the second area 1b of the host 1 includes a reagent pack door configured to be opened from a side of the host 1. An opening behind the reagent pack door and the reagent pack door are configured to accommodate the reagent pack 3. The reagent pack door can be opened by a latch, or other mechanisms in another exemplary embodiment. The reagent pack door can also be located at other positions, for example, the reagent pack door mar he located on either side, back, front or top of the host 1. The latch is adjacent to the reagent pack door for positioning.

In an exemplary embodiment, except for the connecting piece 20 of the reagent pack 3 directly connected to the calibration fluid inlet 6 of the detection card 2, the reagent pack 3 and the host 1 can also be combined through the following methods. The connecting piece 20 of reagent pack 3 is connected to the host sample inlet above one side of host 1, and the host sample inlet is further connected to the calibration liquid inlet 6 of detection card 2 to ensure that the calibration liquid can flow into the detection card 2. The calibration liquid bag 15 of reagent pack 3 includes a bayonet groove, and the bayonet groove is configured to clamp onto the fixing device of reagent pack 3 on one side of host 1, in order to fix the reagent pack 3 in the second area 1b of host 1. The valve connecting piece 22 of reagent pack 3 is connected to the on-off valve control device of the reagent pack 3 on one side of host 1, thereby achieving on-off control of the three-way valve 17 of the reagent pack 3 by host 1. In addition, the pump interface 21 of reagent pack 3 is connected to the peristaltic pump control device on the side of host 1, such as the output shaft of the stepper motor, so that the host 1 can control the peristaltic pump 16 in the reagent pack 3.

In an exemplary embodiment, the host 1 may include one or more ports configured to accommodate a cable or other connection mechanism. The port can he configured to connect the host 1 to other pieces of the system (such as a communication network) or to upload or download information to the host 1. The host 1 can also be configured to exchange data wirelessly, including through Wi Fi (wireless internet technology), another wireless interact connection, or any other wireless information exchange. The host 1 can also include speakers configured to transmit noise or sound response to the user. The host 1 can also include a handle configured to hold the host 1. The handle rotates between two positions depending on whether it is used or not. In exemplary embodiments, the host 1 may also include a support leg configured to allow the host 1 to place on a desktop or other surface. In an exemplary embodiment, the host 1 may also include a barcode scanner installed on the side of host 1. The barcode scanner is configured to scan barcodes on the detection card 2, calibration liquid hag 15, or any other items that have a scannable barcode and are used on the host 1. Barcode scanners can also he configured to scan barcode labels that represent patient or operator identities. In an exemplary embodiment, the barcode scanner emits a beam of light covering the barcode. When the barcode is successfully scanned, the host 1 emits a beep sound and the beam is automatically turn off, When the barcode is not successfully scanned, the host 1 alert the user by making noise or on the display through some other output unit. In exemplary embodiments, the barcode scanner is a one-dimensional barcode scanner. In other embodiments, the barcode scanner is a two-dimensional scanner.

According to an exemplary embodiment, the processing circuit of the host 1 includes an analog-to-digital converter and an analog control board. The analog-to-digital converter is configured to process an analog signal from the photochemical sensor, and transmits the processed digital signal to the analog control board. When the analog control boards herein and in the drawings are designated as “analog control board”, the analog control hoard may include digital processing. Furthermore, the analog control board may utilize the digital-to-analog converter to convert the digital output (turn on/turn off modulated signal) to the analog signal (e.g., for the photochemical sensor).

The processing circuitry and power supply circuitry in the host 1 may he used as independent printed circuit hoard (PCB) integrated on the same PCB, or combined with integration and distribution. The processing circuit and the power supply circuit may include discrete components and/or integrated circuits. For example, the power supply circuit can include all discrete electronic components. The processing circuit may include one or more processors. The processor can he operated in multiple ways as a general-purpose processor, a specialized integrated circuit (ASIC), one or more field programmable gate arrays (FPGA), a set of processing components, or other suitable electronic processing components. The processing circuit may also include one or more memories. The memory can he one or more devices used to store data and/or computer code. The memory can he or include non-transient volatile memory and/or non-volatile memory. The memory may include a database component, an object code component, a script component, or any other type of information structure used to support various activities and the information structure described herein. The memory can be propagatively connected to the processor and includes computer code modules for executing one or more programs described herein.

The optical elements of the host 1 now are described in detail as previously described. In embodiments of the present disclosure, the blood gas parameters in the blood sample are measured by the photochemical sensor, and hemoglobin and its derivatives are detected using optical methods, such as colorimetry without the photochemical sensor. Thus, optical elements are needed to provide the light source, excite the photochemical sensor, transmit the optical signals, and the like.

Specifically, the optical element of the host 1 includes two parts. The first part is arranged in the host 1, and the first part includes a first light source for detecting the hemoglobin and its derivatives and a second light source for detecting the fluid position of the detection card 2. The different detection beams are emitted to detect the presence or absence of fluid in the hemoglobin and its derivatives of the blood sample and in each position monitoring point of the detection card 2, respectively. The second part is arranged in the host 1, and equipped with a light source 24 for optically detecting the blood gas component of the blood sample, as shown in FIG. 12A and FIG. 12B. Specifically, the light source 24 is an excitation light source 24 configured to emit an excitation beam to excite the photochemical sensor in the detection card 2, and the excitation light source is located within the host 1 and is lower in height than the first area 1a.

In an exemplary embodiment, the diagnostic device includes the valve control. mechanism configured to control the valve component of the detection card 2.

The present disclosure has the beneficial effects that the removable detection card for use in in-vitro medical diagnosis device does not store the calibration liquid, so that the removable detection card has low requirement on the storage environment/condition in the transportation and storage link, the use cost of the detection card is greatly reduced, and the calibration precision of the detection card can be ensured at the same time. At the same time, the unique structure design in the detection card, that is, a blood gas detection area and a hemoglobin and its derivatives detection area are simultaneously arranged; makes it possible to complete the detection of blood gas and hemoglobin indexes in one detection card, thereby solving the problem of serious waste of detection card in the prior art. More importantly, the liquid path control area in the detection card is utilized to implement liquid path switching, the same blood sample can be utilized to complete the detection of blood gas and hemoglobin indexes in one detection card, so that the detection blood volume of the patient is greatly reduced; and particularly for the patient with less blood volume, the treatment experience is improved, the treatment cost is greatly reduced, and the detection precision can be ensured.

The terms “generally”, “about”, “substantially”, and similar terms used here have a broad meaning consistent with the generally accepted usage recognized by those skilled in the art, and the subject of this disclosure belongs to the generally accepted usage. Those skilled in the art who review this disclosure should understand that these terms are intended to allow descriptions of certain features described and claimed, without limiting the scope of these features to a set precise numerical range. Therefore, these terms should be interpreted as non-substantive or incoherent modifications or modifications of the subject matter described and claimed priority should be considered within the scope of the present disclosure and explained by the accompanying claims. It should be noted that the term “exemplary” used in this disclosure to describe different embodiments refers to possible examples, illustrations representing and/or possible embodiments (this term does not imply that such embodiments must be extraordinary examples or highest-level examples). The terms “coupled” and “connected” and their analogues used in this disclosure refer to the direct or indirect combination of two components with each other. This combination can be static (such as permanent) or movable (such as removable or releasable). This combination can be achieved by combining two components or integrating the terrain into a single entity with any additional intermediate components, or by combining two components or two components attached to each other with any additional intermediate components. The structure and arrangement of the system and the methods for providing in-vitro medical diagnostic devices as shown in various exemplary embodiments are just illustrated.

only some embodiments have been described in detail in present disclosure, but it will be readily appreciated by those skilled in the art with reference to this disclosure, there may be many modifications (e.g., variations in size, structure, shape and scale of various elements, parameter values, mounting arrangements, use of materials, color, changes in orientation, and so on.) without substantially departing from the novel teachings and advantages of the subject matter disclosed herein. For example, the components shown as a whole can be composed of multiple parts or components, and the position of the components can he reversed or otherwise changed, as well as the nature, quantity, or position of discrete components can be changed. All such modifications are intended to he included in the scope of the present disclosure as defined in the appended claims. The order or sequence of any process or method step can he changed or reordered according to alternative embodiments. The design, operating conditions, and arrangement of various exemplary embodiments can be replaced, modified, altered, and omitted without departing from the scope of the present disclosure.

The diagnostic device is generally shown to include the processing circuitry including the memory. The processing circuitry may include the processor. The processor can he operated as a general-purpose processor, a specialized integrated circuit (ASIC), one or more field programmable gate arrays (FPGA), a set of processing components, or other suitable electronic processing, components. The memory can be one or more devices configured to store data and/or computer code (e.g., RAM, ROM, flash memory, hard disk memory, or the like), to complete and/or implement various programs described in present disclosure. The memory can be or include non-transient volatile memory and/or non-volatile memory. The memory may include the database component, the object code component, the script component, or any other type of information structure used to support various activities and the information structure described herein. The memory can be propagatively connected to the processor and includes computer code modules for executing one or more programs described herein. The foregoing is merely a preferred embodiment of the present disclosure, without limiting the disclosure, any modifications, equivalents, substitutions, and modifications made within the spirit and principles of the disclosure should be included within the scope of the disclosure.

What is claimed is:

1. A removable detection card for an in-vitro medical diagnosis device, comprising:
a blood gas detection area;
a hemoglobin and its derivatives detection area;
an internal pipeline; and
at least two external interfaces comprising a calibration liquid inlet and a sample to be detected inlet, wherein the removable detection card is configured to allow a calibrating liquid required by the removable detection card to enter the removable detection card through the calibration liquid inlet after being mounted in a detection area of an in-vitro medical diagnosis device.

2. The removable detection card as claimed in claim 1, wherein one or more first cavities for blood gas detection is located in the blood gas detection area, and one or more second cavities for detecting a hemoglobin and its derivatives is located in the hemoglobin and its derivatives detection area.

3. The removable detection card as claimed in claim 2, further comprising a photochemical sensor or an electrochemical sensor, wherein the photochemical sensor or the electrochemical sensor are located in the one or more first cavities, and/or the one or more second cavities are configured to detect the hemoglobin and derivatives by colorimetric or electrochemical method.

4. The removable detection card as claimed in claim 2, wherein the second cavity is thinner than the first cavity, and/or a diameter of the second cavity is greater than a diameter of the first cavity; and/or the first cavity has a thickness of 0.6 mm to 0.9 mm, and/or the second cavity has a thickness of 0.1 mm to 0.5 mm; and/or a distance between the one or more first cavities is at least greater than 7 mm, and/or the one or more first cavities have an interior width of 2.5 mm, 3.5 mm. or 4.5 mm; and/or a liquid path width between the one or more first cavities is 1 mm.

5. The removable detection card as claimed in claim 1, further comprising a waste liquid area, wherein the internal pipeline is configured to provide a flow path between the blood gas detection area, the hemoglobin and its derivatives detection area, and the waste liquid area.

6. The removable detection card as claimed in claim 5, further comprising a first control part configured to control on-off between the waste liquid area and the internal and/or a second control part configured to control the on-off between the sample to be detected inlet and the internal pipeline, and/or a third control part configured to control the on-off between the internal pipeline and the hemoglobin and derivatives detection area.

7. The removable detection card as claimed in claim 1, further comprising an elastomeric material and/or valve inside or on a surface of the removable detection card, the elastomeric material and/or valve is configured to implement on-off.

8. The removable detection card as claimed in claim 5, wherein the waste liquid zone has an external interface for communicating with ambient air pressure, and/or the external interfaces are configured to he distributed on the same or different sides of the removable detection card.

9. The removable detection card as claimed in claim 5, wherein the detection card comprises at least two control parts; in an initial state, the two control parts are configured to enable the pipelines corresponding, to the two control parts to he in a cut-off state; and after the removable detection card enters an operation state, the two control parts are configured to enable the pipelines corresponding to only one control part to be in a conduction state at the same time.

10. The removable; detection card as claimed in claim 9, wherein a first control part controls on-off between the waste liquid area and the internal pipeline in response to calibrating the sensor in the detection card or requiring discharge of the calibration liquid to the waste liquid area.

11. The removable detection card as claimed in claim 10, wherein a second control part controls on-off between the sample to be detected inlet and the internal pipeline in response to injecting a sample to be detected into the removable detection card.

12. The removable detection card as claimed in claim 11, wherein a third control part controls on-off between the internal pipeline and the hemoglobin and its derivatives detection area in response to transferring the sample to be detected in the blood gas detection area to the hemoglobin and its derivatives detection area; and/or each of the first control part, the second control part and the third control part in the removable detection card controls on-off of a corresponding, pipeline in response to a power drive in a host of the in-vitro medical diagnosis device.

13. A detection method for a removable detection card, comprising:
providing a removable detection card comprising a control unit, wherein an internal pipeline is configured to provide a flow path between a blood gas detection area and a hemoglobin and its derivatives detection areas;
in response to a first state of the control unit, a first sample to be detected enters the blood gas detection area; and
in response to a second state of the control unit, a second sample to be detected enters the hemoglobin and its derivatives detection area.

14. The detection method as claimed in claim 13, wherein the first sample to he detected and the second sample to be detected are the same sample to be detected; and/or the removable detection card further comprises a waste liquor area, and the internal pipeline is configured to provide the flow path between the blood gas detection area, the waste liquor zone, and the hemoglobin and its derivatives detection area.

15. The detection method as claimed in claim 14, further comprising: in response to a third state of the control unit, a calibration liquid enters the blood gas detection area for sensor calibration, and the calibration liquid is transferred to the waste liquid area after completing calibration.

16. The detection method as claimed in claim 14, wherein the second sample to be detected. enters the hemoglobin and its derivatives detection area, and the second sample to be detected is transferred to the waste liquid area after the hemoglobin and its derivatives detection is completed.

17. The detection method as claimed in claim 13, wherein the control unit comprises at least three control parts configured to be driven by a host of an in-vitro medical diagnosis device; and a first control part of the at least three control parts controls on-off between the waste liquid area and the internal pipeline of the removable detection card, a second control part controls on-off between a sample to he detected inlet and the internal pipeline, and a third control part controls the on-off between the internal pipeline and the hemoglobin and its derivatives detection area.

18. The detection method as claimed in claim 17, wherein the first state is that the second control part is in conduction, and the first control part and the third control part are both in turn-off state, to make the sample to be detected inlet and the internal pipeline be conducted; and the removable detection card is configured to be provided with a negative pressure by a reagent pack that is connected to the removable detection card through a calibration liquid inlet, to suck the sample to be detected into the blood gas detection area.

19. The detection method as claimed in claim 17, wherein the second state is that the third control part is in conduction, and the first control part and the second control part are in turn-off state, so that the internal pipeline is communicated with the hemoglobin and its derivatives detection area; and the removable detection card is configured to he provided with a positive pressure by a reagent pack that is connected to the removable detection card through a calibration liquid inlet, to transfer the sample to he detected in the blood gas detection area to the hemoglobin and its derivatives detection area.

20. The detection method as claimed in claim 17, wherein the control unit has a third state, the third state is that the first control part is in conduction, and the first control part and the second control part are both in the off state, so that the waste liquid area is communicated with the internal pipeline; and the removable detection card is configured to be provided with a positive pressure by a reagent pack that is connected to the removable detection card through a calibration liquid inlet, the calibrating liquid in the reagent pack is transferred to the blood gas detection area through the calibration liquid inlet, and after calibration, the reagent pack provides the positive pressure again to transfer the calibrating liquid in the blood gas detection area to the waste liquid area.
